(19) United States
(12) Patent Application Publication
(10) Pub. No.: US 2026/0293828 A1
(43) Pub. Date: Oct. 1, 2026

(54) Brussels Sprouts, Headed Cabbage, Cauliflower and Kale Plants with a Non-Bitter Taste

(71) Applicant: Bejo Zaden B.V., Warmenhuizen (NL)

(72) Inventors: Jan Sybe Wijngaarden, Warmenhuizen (NL); Johannes Gerardus Maria Hoogland, Warmenhuizen (NL); Johannes Theodorus Wilhelmus Ligthart, Warmenhuizen (NL); Marcel Adriaanse, Warmenhuizen (NL); Stefanus Johannes Kaandorp, Warmenhuizen (NL); Roelof Marinus Veenstra, Warmenhuizen (NL); Albertus Johannes Maria Schrijver, Warmenhuizen (NL)

(21) Appl. No.: 19/480,694

(22) PCT Filed: May 2, 2023
(86) PCT No.: PCT/EP2023/061506

§ 371 Date: Oct. 31, 2025

(51) Int. Cl. A01H 1/00; A01H 5/12; A01H 6/20; C12N 9/02

(52) U.S. Cl. CPC A01H 1/101; A01H 5/12; A01H 6/203; C12N 9/0071

(57) ABSTRACT

Provided herein are Brussels sprout, kale, cauliflower or cabbage plants substantially not, or not, including sinigrin and progoitrin. Also provided herein are non-bitter tasting Brussels sprout, kale, cauliflower or cabbage plants. The present invention further relates to seeds, progeny, edible parts, egg cells, callus, suspension culture, somatic embryos, clones, embryos, or plant parts, or cells of the present plants and methods for providing or selecting the present plants. Specifically, the present invention relates to plants, wherein the genome of the plants includes a gene encoding an enzymatically inactive 2-oxa-acid dependent dioxygenase (ALK) protein.

### CROSS-REFERENCE TO RELATED APPLICATION

This application is the United States national phase of International Patent Application No. PCT/EP2023/061506 filed May 2, 2023, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2505696.xml. The size of the file is 39,094 bytes, and the file was created on Oct. 30, 2025.

### BACKGROUND OF THE INVENTION

### Field of the Invention

The present invention relates to Brussels sprout, kale, cauliflower or cabbage plants substantially not, or not, comprising sinigrin and progoitrin. Formulated differently, the present invention relates to non-bitter tasting Brussels sprout, kale, cauliflower or cabbage plants. The present invention further relates to seeds, progeny, edible parts, egg cells, callus, suspension culture, somatic embryos, clones, embryos, or plant parts, or cells of the present plants and methods for providing or selecting the present plants.

Brassica oleracea is a member of the Brassicaceae family or crucifers (Cruciferae). This species has many cultivars, which encompass several food crops. The most important of these cultivars are Brassica oleracea var. acephala (kale); Brassica oleracea var. alboglabra, (Chinese kale); Brassica oleracea var. botrytris (cauliflower and Romanesco broccoli); Brassica oleracea var. capitata (red and white cabbage, Savoy cabbage); Brassica oleracea var. gemmifera (Brussels sprouts); Brassica oleracea var. gongylodes (kohlrabi) and Brassica oleracea var. italica (broccoli); Each of these cultivars has been selected and is cultivated for a specific part of the plant. For example, white and red cabbage have prominent leaves, Brussels sprouts are formed by the axial buds of the Brassica plant, while cauliflower is white inflorescence meristem, and broccoli is formed by the flower head of the plant.

Brussels sprouts are typically 1.5-4.0 cm in diameter and resemble miniature cabbages. They comprise fresh and tender lobules formed in axillary buds of edible plants, and the lobules are soft in texture, rich in taste, unique in flavor, and easy to refrigerate and keep fresh.

Brussels sprouts grow in temperature ranges of 7-24° C. and are ready for harvest 3 to 6 months after planting. The edible sprouts grow like buds in helical patterns along the side of long, thick stalks. Sprouts may be picked by hand or mechanically depending on the variety. Harvest season in temperate zones of the northern latitudes is September to March, making Brussels sprouts a traditional winter-stock vegetable.

In Europe, Brussels sprouts are predominantly cultivated in the Netherlands, UK, France, Germany and Belgium. They are also cultivated in Mexico, USA and in China. Brussels sprouts are a rich source of vitamins C and K as well as B1, B6, folate and minerals. They are suitable for diets, since they regulate the intestine and the excretion of the body fluids; they are also low in calories and a good source of protein (3.5 g protein/100 gr raw vegetable). Additionally, Brussels sprouts have anticarcinogenic properties.

Brussels sprouts are highly appreciated for their cooking properties and particular taste, similar to the rest of the cabbages. They are typically cooked by boiling, steaming, stir frying, grilling or roasting. A generally undesirable characteristic of Brussels sprouts is bitterness. Albeit bitterness is appreciated by some, it is disliked by many consumers, mostly children. This creates a need to provide Brussels sprouts that are less, or not, bitter.

The cultivar headed cabbage has a wide phenotypic range. There are more flat varieties but also rounded and pointy-headed cabbages. Their shape can be bred according to the demand of a given market. The demand for a particular shape of the headed cabbage can change over time. Previously, markets generally preferred headed cabbage with large heads, while currently smaller cabbages are in demand. Beyond shape, there is a rather large variation in cultivation days till harvest between varieties. Early cultivars and varieties mature in 60 days, while late types need 150 growing days. Varieties can also vary in leaf position, waxiness of the heads, the size of the head, the shape of the head, and internal core length. Headed cabbage is a rich source of nutrients such as vitamins B1, B6, folate, vitamin C, vitamin K and to a lesser extent the vitamins B2 and B3. Headed cabbage contains glucosinolates and glycosides. These compounds significantly contribute to the characteristic taste of headed cabbage.

Cauliflower is cultivated in different climates ranging from Northern Europe to India. Plants for commercial production are typically started in a greenhouse from seeds. After about 35 to 40 days cauliflower seedlings are transplanted to the field, where they are grown for another 40-300 days, depending on variety and local growing conditions, to maturity and harvest. During the curd initiation phase the meristem of the cauliflower plant forms a generative bud; this bud will develop into the mature flower head, or curd, which is the harvested and marketed part of the plant. Some cauliflower varieties require vernalization. Typically, only the head is eaten—the edible white flesh is sometimes called “curd”. When cauliflower is mature, heads appear clear white, compact, and 15-20 cm in diameter. Next to the traditional white cauliflower, there are orange, green and purple varieties. Cauliflower is a source of carbohydrates, fiber and protein and contains negligible fat, as well as vitamins B, C and K. Cauliflower contains several non-nutrient phytochemicals common in the cabbage family including isothiocyanates and glucosinolates.

Kale, also known as leaf cabbage, belongs to a group of cabbage cultivars grown for their edible leaves, although some are used as ornamentals. Kale plants have green or purple leaves, and the central leaves do not form a head (as with headed cabbage). Kales are considered to be closer to wild cabbage than most of the many domesticated forms of Brassica oleracea. Kale is a hardy vegetable and thrives in wintertime, surviving in temperatures as low as −15.0° C., Celsius. Kale can become sweeter after a heavy frost. The nutrient rich kale is a source of carbohydrates, proteins and fat as well as vitamins, including K, C, A, E and to a lesser extents group B vitamins and dietary relevant minerals: manganese, magnesium, potassium, iron, phosphorous and calcium.

The phytochemicals found in kale include the carotenoids lutein and zeaxanthin and polyphenols such as ferulic acid. As with other Brassica cultivars, kale contains glucosinolate compounds (e.g. glucoraphanin) that contribute to its overall taste.

Glucosinolates (GLS) constitute a class of organic compounds that contain sulfur and nitrogen and are derived from glucose and an amino acid. There are at least 132 known chemical structures and these compounds are mainly found in cruciferous plants. GLS and mostly their enzymatic hydrolysis products are versatile bioactive compounds. They can have negative effects: e.g., attracting diamondback moth, a pest of cruciferous plants, which is able to recognize the presence of GLS, allowing it to identify the proper host plant or progoitrin contributing to goiter disease in cattle. They also have positive roles such as anticarcinogenic (sulforaphane), antioxidant and antifungal effect in humans or deterring or toxic activity for some insects (bioinsecticides). Biosynthesis process of these compounds is very complex but also well studied in Arabidopsis and Brassica species.

Based on the amino acid glucosinolates are derived from, these compounds can be divided into three classes: (1) aliphatic GSLs derived from alanine (Ala), leucine (Leu), isoleucine (Ileu), valine (Val), and methionine (Met); (2) aromatic GSLs derived from phenylalanine (Phe) and tyrosine (Tyr) and (3) indolic GSLs derived from tryptophan (Trp). Aliphatic GSL are the most abundant group and in Brassica crops most GLS are synthesized from methionine.

In all three groups, the biosynthetic pathway has three main phases: elongation of amino acids by insertion of methylene groups into their side chains, followed by reconfiguration of the amino acid moiety to yield a core structure of the glucosinolate and lastly modification stage where secondary transformations take place that result in the final structure of the glucosinolate.

Aliphatic pathways typically starts with methionine, which is deaminated to the corresponding 2-oxo acid by a branched chain amino acid aminotransferase (BCAT). Then, a sequential addition of single methylene groups to the side chain of the 2-oxo acid takes place in three reactions involving methylthiolalkylmalate (MAM), an isopropylmalate isomerase (IPMI), and isopropylmalate dehydrogenase (IPMDH) yielding an elongated 2-oxo-acid. Subsequently the elongated 2-oxo-acid can either proceed through another elongation round or can be transaminated by a BCAT to the corresponding chain elongated amino acid.

Proceeding to the second stage of the biosynthesis, cytochrome P450 (CYP79s) catalyzes the conversion of the amino acid derivatives to aldoximes, oxidized later by CYP83s into nitrile oxides, transformed to thiohydroximates via glutathione (GSH)S-transferases (GSTFs) and the C-S lyase (SUR1) reaction, and finally converted to the GLS core structure by S-glucosyltransferases (UGT74s) and sulfotransferases (SOTs).

In parallel, GSH is produced by the action of specific ligases (GSH1 and GSH2) from cysteine, glutamate, and glycine and ensures sulfur supply for GLS biosynthesis, yielding GSH conjugates. Alkenylation, benzoylation, glycosylation, hydroxylation, methoxylation, oxygenation, and sulfonation may affect the elongated amino acid, increasing the chemical structure diversity of GLS. The methylthioalkyl GLS are S-oxygenated in a reaction catalyzed by flavin monooxygenases (FMOGS-OXs) to methylsulfinylalkyl GLS, such as 4-methylsulfinylbutyl-GL (Glucoraphanin) or 4-methylsulfinylpropyl (Glucoiberin). Glucoiberin and Glucoraphanin are substrates for 2-oxa acid dependent dioxygenase (AOP2 or ALK). The AOP2 gene product converts these methylsulfinylalkyls into alkenyl glucosinoplates. In the case of the conversion of glucoiberin the final product of the pathway is sinigrin. The final product from glucoraphanin is progoitrin.

Human perception of taste is complex and influenced by many factors. The bitter taste in Brassica cultivars, especially Brussels sprouts, is generally associated with the glucosinolates sinigrin and progoitrin. Van Dorn et al. (J Sci Food Agric; 1998, 78, 30-38) have demonstrated that there is a linear correlation in the perception of bitterness in consumers of Brussels sprouts and increased content of sinigrin and progoitrin in this vegetable. Brussels sprouts with high levels of sinigrin and progoitrin are often considered too bitter and, accordingly, of poor taste.

### Description of Related Art

There have been previous breeding efforts to create less bitter tasting sprouts. These involved crossing of varieties that already exhibited a milder taste and selection for offspring with an even more mild, less bitter taste. However, the resulting Brussels sprouts still had significant levels of sinigrin and progoitrin.

Considering the above, there is a need, or desire, in the art to provide Brussels sprouts that completely, or substantially, do not produce sinigrin and progoitrin. Because headed cabbage, cauliflower and kale contain glucosinolates that influence their taste, there is a further need, or desire, in the art to provide headed cabbage, cauliflower and kale that completely, or substantially, do not produce sinigrin and progoitrin.

### SUMMARY OF THE INVENTION

It is an object of the present invention, amongst other objects to meet the above need, or desire, in the art.

According to the present invention, this need, or desire in the art, is met as outlined in the appended claims.

Specifically, this need, or desire, in the art is met by providing plants, wherein the plants substantially do not, or do not, comprise sinigrin and progoitrin, the genome of the plants comprise a gene encoding an enzymatically inactive 2-oxa-acid dependent dioxygenase (ALK) protein, the enzymatically inactive ALK protein comprises one or more mutations as compared to
SEQ ID No. 1 wherein the plant is Brussels sprout; or
SEQ ID No. 2 wherein the plant is kale or cauliflower; or
SEQ ID No. 3 wherein the plant is cabbage.

Alternatively, this need, or desire, in the art is met by providing plants, wherein the plants substantially do not, or do not, comprise sinigrin and progoitrin, the genome of the plants comprise a gene encoding an enzymatically inactive 2-oxa-acid dependent dioxygenase (ALK) protein, the enzymatically inactive ALK protein encoding gene comprises one or more mutations as compared to
SEQ ID No. 11 wherein the plant is Brussels sprout; or
SEQ ID No. 12 wherein the plant is kale or cauliflower; or
SEQ ID No. 13 wherein the plant is cabbage.

Within the context of the present invention, plants that do not produce sinigrin and progoitrin, are plants for which the level of sinigrin and progoitrin is under the detection limit, i.e., less than 3 micromole progoitrin or sinigrin/100 grams fresh weight as measured with LC-MS, using standards of sinigrin and progoitrin for identification and quantification of LC-MS peaks, as defined in detail below (Example 2).

Within the context of the present invention, plants that substantially do not produce sinigrin and progoitrin, are plants for which the sum of sinigrin and progoitrin is not more than 10 micromole/100 gram fresh weight, as measured with LC-MS, using standards of sinigrin and progoitrin for identification and quantification of LC-MS peaks, as defined in detail below (Example 2).

The present inventors produced the above plants using various classical breeding and molecular biology based techniques known in the art such as (1) classical breeding involving crossing and selection as well as (2) genome editing technology, namely CRISPR/Cas9, where a mutation was introduced in the coding sequence of the AOP2 (ALK). In both cases the resulting plants had a defective AOP2 (ALK) gene product.

### DESCRIPTION OF THE INVENTION

According to a preferred embodiment, the present invention relates to the above plants, wherein the one or more mutations are selected from the group consisting of insertions, deletions, frame shifts, substitutions, and truncations in
SEQ ID No. 1 wherein the plant is Brussels sprout; or
SEQ ID No. 2 wherein the plant is kale or cauliflower; or
SEQ ID No. 3 wherein the plant is cabbage.

Alternatively, according to another preferred embodiment, the present invention relates to the above plants, wherein the one or more mutations are selected from the group consisting of insertions, deletions, frame shifts, substitutions, and truncations in
SEQ ID No. 11 wherein the plant is Brussels sprout; or
SEQ ID No. 12 wherein the plant is kale or cauliflower; or
SEQ ID No. 13 wherein the plant is cabbage.

According to another preferred embodiment, the present gene encoding an enzymatically inactive 2-oxa-acid dependent dioxygenase (ALK) protein, said enzymatically inactive ALK protein is:
SEQ ID No. 16, or amino acid sequences having at least 90% sequence identity therewith, wherein the plant is Brussels sprout; or
SEQ ID No. 17, or amino acid sequences having at least 90% sequence identity therewith, wherein the plant is kale or cauliflower; or
SEQ ID No. 18, or amino acid sequences having at least 90% sequence identity therewith, wherein the plant is cabbage.

Within the context of the present invention, at least 90% sequence identity comprises at least 91%, 92%, 93%, 94%, 95%, 96%, 97%, 98% or 99% sequence identity.

According to an especially preferred embodiment, the present gene encoding an enzymatically inactive 2-oxa-acid dependent dioxygenase (ALK) protein is homozygously present in the genome of the above plants.

According to another especially preferred embodiment, the present plants do not comprise a gene encoding an enzymatically active 2-oxa-acid dependent dioxygenase (ALK) protein of:
SEQ ID No. 1 wherein the plant is Brussels sprout; or
SEQ ID No. 2 wherein the plant is kale or cauliflower; or
SEQ ID No. 3 wherein the plant is cabbage.

According to a particularly preferred embodiment, the present gene encoding an enzymatically inactive 2-oxa-acid dependent dioxygenase (ALK) protein is obtainable from, obtained from, or is from a plant whereof representative seeds are deposited under deposit number NCIMB 44053 (Oct. 5, 2022, Wellheads Pl, Dyce, Aberdeen AB21 7 GB, United Kingdom).

The present plants are preferably cytoplasmic male sterile (CMS) plants and/or hybrid plants.

Considering the beneficial properties of the plants described above, the present invention also relates to seeds, progeny, edible parts, egg cells, callus, suspension culture, somatic embryos, clones, embryos, or plant parts, or cells of the present plants.

The present plants can be suitably provided by methods comprising the step of introducing one or more mutations in a gene encoding an enzymatically active 2-oxa-acid dependent dioxygenase (ALK) protein to provide a plant that substantially does not comprise sinigrin and progoitrin. Preferably, the one or more mutations in the gene encoding an enzymatically active 2-oxa-acid dependent dioxygenase (ALK) protein are provided by genome editing, CRISPR Cas or mutagenesis.

Alternatively, the present plants can be suitably provided by methods comprising the steps of:
i) providing a Brassica plant comprising a gene encoding an enzymatically active 2-oxa-acid dependent dioxygenase (ALK) protein;
ii) crossing the plant of step (i) with a Brassica plant comprising a gene encoding an enzymatically inactive 2-oxa-acid dependent dioxygenase (ALK) protein;
iii) optionally, selfing the plant obtained in step ii) at least once;
iv) selecting a plant homozygously comprising in its genome a gene encoding an enzymatically inactive 2-oxa-acid dependent dioxygenase (ALK) protein.

According to the present invention, the Brassica plant comprising a gene encoding an enzymatically inactive 2-oxa-acid dependent dioxygenase (ALK) protein of step (ii) is, or is preferably derived from, a plant whereof representative seeds are deposited under deposit number NCIMB 44053.

According to the present invention, based on the sequences provide herein below, the present invention also relates to:
Use of a gene encoding an enzymatically inactive 2-oxa-acid dependent dioxygenase (ALK) protein as defined above for providing Brussels sprouts, kale, cauliflower or cabbage plants that substantially do not, or do not, comprise sinigrin and progoitrin;
Use of a gene encoding an enzymatically inactive 2-oxa-acid dependent dioxygenase (ALK) protein as defined above for providing Brussels sprouts, kale, cauliflower or cabbage plants with less bitter taste; or
Use of a gene encoding an enzymatically inactive 2-oxa-acid dependent dioxygenase (ALK) protein as defined above, for selecting Brussels sprouts, kale, cauliflower or cabbage plants that substantially, or do not, comprise sinigrin and progoitrin.

The present invention will be further detailed below in examples of different embodiments of the present invention.

### Examples

### Deposit Information

Representative seeds of plants according to the invention, Brussels sprouts, Brassica oleracea var gemmifera, plants F 19000, deposit NCIMB 44053, were deposited at NCIMB Limited, Craibstone Estate, 35 Ferguson Building, Bucksburn, Aberdeen AB21 9YA, United Kingdom on 5th October 2022 (present address Wellheads Pl, Dyce, Aberdeen AB21 7 GB, United Kingdom).

### Example 1: Development of Non-Bitter Brussels Sprouts

For the development of a non-bitter Brussels sprout, a cross was made between the Brassica line containing a non-functional ALK gene and an Brussels sprouts inbred line. Subsequently, the F1 was repeatedly backcrossed with the Brussels sprouts line. In the first two backcross generations, a plant with a unique crossover-event was selected using molecular markers. In successive generations plants were selected based on genome-wide recurrency. The final selected backcross plant was recurrent for all chromosomes but contained a small introgression with the non-functional ALK gene. This plant was inbred and a plant homozygous for the novel introgression was selected. Seeds were harvested and the new line was morphologically identical to the original Brussels sprouts line in a field trial with the exception that the newly created Brussels sprout was not bitter. Moreover, a taste test was performed to validate the effect of the introgression with the non-functional ALK gene, see Example 4.

TABLE 1
Sequences of Alk Proteins.

| | | |
|---|---|---|
| Sprout | Seq | MGADTPQLPVIYLSDQTLKPGGEKWVEVRSDVRKALEDYGAFEVSYDRV |
| | ID | SEELKKSVLDAMIELFELPVEAKQRNVSPKPYTGYSTHNGLSESLGIQDSN |
| | No | VLEKVNEFTQLLRPDCEGNKTMSETIQKFSEKLAELDVMVRRMVIESFGIE |
| | 1 | NYFDEHLKSTNYRLRLMKYVAPPDVDANVAVSTKDSVDGANTNTTANA |
| | | DAGDIANGIAKVHIDDDANAKGDVCTGVGTNHNGDDVNTGDCANVKSN |
| | | VDVGTNVSAKSSVGADVNIGTIADVKANTGTRTSATVGVSDSVKANGGA |
| | | DDEEKKLGLPSHTDKNLLTVLYQYEIEGLEVLTKDEKWIRLKPSHNSFVV |
| | | MAGDSLYALMNGRLSRPFHRVRVTERKKTRYSIALFSTPNGDYIIEPPKEL |
| | | VDEKHPRLFKPFTFVDLMSFYHTEAGRRPRSTLHAYCAVSGA |
| Kale/ | Seq | MGADTPQLPVIYLSDQTLKPGGEKWVEVRSDVRKALEDYGAFEVSYDRV |
| Cauliflower | ID | SEELKKSVLDAMIELFELPVEAKQRNVSPKPYTGYSTHNGLSESLGIQDSN |
| | No | VLEKVNEFTQLLRPDCEGNKTMSETIQKFSEKLAELDVMVRRMVIESFGIE |
| | 2 | NYFDEHLKSTNYRLRLMKYVAPPDVDANVAVSTKDSVDGANTNTTANA |
| | | DAGDIANGIAKVHIDDDANAKGDVCTGVGTNHN |
| | | GDDVNTGDCANVKSNVDVGTNVSAKSSVGADVNIGTIADVKANTGTRTS |
| | | ATVGVSDSVKANGGADDEEKKLGLPSHTDKNLLTVLYQYEIEGLEVLTKD |
| | | EKWIRLKPSHNSFVVMAGDSLYALMNGRLSRPFHRVRVTERKKTRYSIAL |
| | | FSTPNGDYIIEPPKELVDEKHPRLFKPFTYVDLMSFYHTEAGRRPRSTLHAY |
| | | CTVSGA |
| Cabbage | Seq | MGADTPQLPVIYLSDQTLKPGSEKWVEVRSDVRKALEDYGGFEVSYDRV |
| | ID | SEELKKSVLDAMIELFELPVEAKQRNVSPKPYTGYSTHNGLSESLGIQDSN |
| | No | VLEKVNEFTQLLRPDCEGNKTMSETIQKFSEKLAELDVMVRRMVIESFGIE |
| | 3 | NYFDEHLKSTNYRLRLMKYVAPPDVDANVAVSTKDSVDGANTNTTANA |
| | | DAGDIANGIAKVHIDDDANAKGDVCTGVGTNHNGDDVNTGDCANVKSN |
| | | VDVGTNVSAKSSVGADVNIGTIADVKANTGTRTSANVGVSDSVKANGGA |
| | | DDEEKKLGLPSHTDKNLLTVLYQYEIEGLEVLTKDEKWIRLKPSHNSFVV |
| | | MAGDSLYALMNGRLSRPFHRVRVTERKKTRYSIALFSTPNGDYIIEPPKEL |
| | | VDEKHPRLFKPFTFVDLMSFYHTEAGRRPRSTLHAYCAVSGA |
| Non- | Seq | MGADTPQLPVIYLSDQTLKPGGEKWVEVRSDVRKALEDYGAFEVSYDRV |
| functional | ID | SEELKKSVLDAMIELFELPVEAKQRNVSPKPYTGYSTHNGLSESLGIQDSN |
| 1 | No | VLEKVNEFTQLLRPDCEGNKTMSETIQKFSEKLAELDVMVRRMVIESFGIE |
| | 4 | NYFDEHLKSTNYRLRLMKYVAPPDVDANVAVSTKDSVDGANTNTTANA |
| | | DAGDIANGIAKVHIDDDANAKGDVCTGVGTNHNGDDVNTGDCANVKSN |
| | | VDVGTNVSAKSSVGADVNIGTIADVKANTGTRTSANVGVSDSVKANGGA |
| | | DDEEKKLGLPSHTDKPFDGALSI |
| Non- | Seq | MGADTPQLPVIYLSDQTLKPGSEKWVEVRSDVRKALEDYGAFEVSYDRV |
| functional | ID | SEELKKSVLDAMIELFELPVEAKQRNVSPKPYTGYSTHNGLSESLGIQDSN |
| 2 | No | VLEKVNEFTQLLRPDCEGNKTMSETIQKFSEKLAELDVMVRRMVIESFGIE |
| | 5 | NYFDEHLKSTNYRLRLMKYVAPPDVDANVAVSTKDSVDGANTNTTANA |
| | | DAGDIANGIAKVHIDDDANAKGDVCTGVGTNHNGDDVNTGDCANVKSN |
| | | VDVGTNVSAKSSVGADVNIGTIADVKANTGTRTSANVGVSDSVKANGGA |
| | | DDEEKKLGLPSHTDKPFDGALSI |

TABLE 2
cDNA sequences for Alk proteins.

| | | |
|---|---|---|
| Sprout | Seq | Atgggtgcagacactcctcaacttccagtcatctatctctcggaccaaaccctaaaaccaggaggtgagaagtgggttgaagtgagga |
| | ID | gtgatgtccgtaaagctcttgaagactacggcgcttttgaggtgtcatatgatagagtgtcagaagagcttaagaagtcagttttggatgc |
| | No | catgatagagcttttcgagttaccagttgaggctaaacagagaaacgtgtctccgaaaccttacactggatattcaactcataatggtctct |
| | 6 | ctgagagtctagggatccaggattccaatgttttggagaaagttaacgaatttactcaactgctacgccctgattgtgagggtaacaagac |
| | | tatgagcgaaacgatccagaagttttcagagaagttagcagaattggatgtaatggtgagaagaatggtcatagagagctttgggatag |
| | | aaaactactttgatgaacacctcaagtcaacgaattaccgtttgcgactgatgaagtatgtagcaccacctgatgttgatgctaatgttgcg |
| | | gtcagtactaaagattctgttgatggtgctaatacgaatacaactgctaacgctgatgctggtgatattgctaatggtattgctaaagttcata |
| | | ttgatgacgatgctaatgccaagggtgacgtttgcactggtgttggtaccaatcataatggtgatgacgttaatactggtgattgtgctaatg |
| | | ttaagtctaatgtcgacgttggtactaatgttagt |
| | | Gctaaatctagtgttggtgctgatgttaacattggcactattgctgatgttaaggctaatactggtactcgtactagtgctactgttggcgtta |
| | | gtgatagtgttaaagctaatggtggcgcagatgatgaggagaagaaattaggtctaccttctcatactgataaaaaccttttgacggtgctt |
| | | tatcaatatgagattgaaggtttggaggtgttaaccaaagatgaaaagtggatcaggttaaaaccatctcataactctttcgttgttatggct |
| | | ggagattctctatatgcacttatgaatggtagactatctcgtccgtttcatagagtaagagtaacggaaagaaagaagacaaggtattcaa |
| | | tagcattgttctcgactccaaacggagattacatcatcgaaccaccaaaagagcttgtggacgagaagcacccacgtctcttcaaaccat |
| | | tcactttcgttgacttgatgagtttctatcacactgaggctggtcgtagacctcgttctactcttcacgcttattgtgccgtctctggagcataa |
| Kale | Seq | atgggtgcagacactcctcaacttccagtcatctatctctcggaccaaaccctaaaaccaggaggtgagaagtgggttgaagtgagga |
| Cauli- | ID | gtgatgtccgtaaagctcttgaagactacggcgcttttgaggtgtcatatgatagagtgtcagaagagcttaagaagtcagttttggatgc |
| flower | No | catgatagagcttttcgagttaccagttgaggctaaacagagaaacgtgtctccgaaaccttacactggatattcaactcataatggtctct |
| | 7 | ctgagagtctagggatccaggattccaatgttttggagaaagttaacgaatttactcaactgctacgccctgattgtgagggtaacaagac |
| | | tatgagcgaaacgatccagaagttttcagagaagttagcagaattggatgtaatggtgagaagaatggtcatagagagctttgggatag |
| | | aaaactactttgatgaacacctcaagtcaacgaattaccgtttgcgactgatgaagtatgtagcaccacctgatgttgatgctaatgttgcg |
| | | gtcagtactaaagattctgttgatggtgctaatacgaatacaactgctaacgctgatgctggtgatattgctaatggtattgctaaagttcata |
| | | ttgatgacgatgctaatgccaagggtgacgtttgcactggtgttggtaccaatcataatggtgatgacgttaatactggtgattgtgctaatg |
| | | ttaagtctaatgtcgacgttggtactaatgttagt |
| | | gctaaatctagtgttggtgctgatgttaacattggcactattgctgatgttaaggctaatactggtactcgtactagtgctactgttggcgtta |
| | | gtgatagtgttaaagctaatggtggcgcagatgatgaggagaagaaattaggtctaccttctcatactgataaaaaccttttgacggtactt |
| | | tatcaatatgagattgaaggtttggaggtgttaaccaaagatgaaaagtggatcaggttaaaaccatctcataactctttcgttgttatggct |
| | | ggagattctctatatgcacttatgaatggtagactatctcgtccgtttcatagagtaagagtaacggaaagaaagaagacaaggtattcaa |
| | | tagcattgttctcgactccaaacggagattacatcatcgaaccaccaaaagagcttgtggacgagaagcacccacgtctcttcaaaccat |
| | | tcacttacgttgacttgatgagtttctatcacactgaggctggtcgtagacctcgttctactcttcacgcttattgtaccgtctctggagcataa |
| Cabbage | Seq | atgggtgcagacactcctcaacttccagtcatctatctctcggaccaaaccctaaaaccaggaagtgagaagtgggttgaagtgaggag |
| | ID | tgatgtccgtaaagctcttgaagactacggcggttttgaggtgtcatatgatagagtgtcagaggagcttaagaagtcagttttggatgcc |
| | No | atgatagagcttttcgagttaccagttgaggctaaacagagaaacgtgtctccgaaaccgtacactggatattcaactcataatggtctctc |
| | 8 | tgagagtctagggatccaggattccaatgttttggagaaagttaacgaatttactcaactgctacgccctgattgtgagggtaacaagact |
| | | atgagcgaaacgatccagaagttttcagagaagttagcagaattggatgtaatggtgagaagaatggtcatagagagctttgggataga |
| | | aaactactttgatgaacacctcaagtcaacgaattaccgtttgcgactgatgaagtatgtagcaccacctgatgttgatgctaatgttgcgg |
| | | tcagtactaaagattctgttgatggtgctaatacgaatacaactgctaacgctgatgctggtgatattgctaatggtattgctaaagttcatatt |
| | | gatgacgatgctaatgccaagggtgacgtttgcactggtgttggtaccaatcataatggtgatgacgttaatactggtgattgtgctaatgtt |
| | | aagtctaatgtcgacgttggtactaatgttagt |
| | | gctaaatctagtgttggtgctgatgttaacattggcactattgctgatgttaaggctaatactggtactcgtactagtgctaatgttggcgtta |
| | | gtgatagtgttaaagctaatggtggcgcagatgatgaggagaagaaattaggtctaccttctcatactgataaaaaccttttgacggtgctt |
| | | tatcaatatgagattgaaggtttggaggtgttaaccaaagatgaaaagtggatcaggttaaaaccatctcataactctttcgttgttatggct |
| | | ggagattctctatatgcacttatgaatggtagactatctcgtccgtttcatagagtaagagtaacggaaagaaagaagacaaggtattcaa |
| | | tagcattgttctcgactccaaacggagattacatcatcgaaccaccaaaagagcttgtggacgagaagcacccacgtctcttcaaaccat |
| | | tcactttcgttgacttgatgagtttctatcacactgaggctggtcgtagacctcgttctactcttcacgcttattgtgccgtctctggagcataa |
| Non- | Seq | atgggtgcagacactcctcaacttccagtcatctatctctcggaccaaaccctaaaaccaggaggtgagaagtgggttgaagtgagga |
| func- | ID | gtgatgtccgtaaagctcttgaagactacggcgcttttgaggtgtcatatgatagagtgtcagaagagcttaagaagtcagttttggatgc |
| tional | No | catgatagagcttttcgagttaccagttgaggctaaacagagaaacgtgtctccgaaaccttacactggatattcaactcataatggtctct |
| 1 | 9 | ctgagagtctagggatccaggattccaatgttttggagaaagttaacgaatttactcaactgctacgccctgattgtgagggtaacaagac |
| | | tatgagcgaaacgatccagaagttttcagagaagttagcagaattggatgtaatggtgagaagaatggtcatagagagctttgggatag |
| | | aaaactactttgatgaacacctcaagtcaacgaattaccgtttgcgactgatgaagtatgtagcaccacctgatgttgatgctaatgttgcg |
| | | gtcagtactaaagattctgttgatggtgctaatacgaatacaactgctaacgctgatgctggtgatattgctaatggtattgctaaagttcata |
| | | ttgatgacgatgctaatgccaagggtgacgtttgcactggtgttggtaccaatcataatggtgatgacgttaatactggtgattgtgctaatg |
| | | ttaagtctaatgtcgacgttggtactaatgttagt |
| | | gctaaatctagtgttggtgctgatgttaacattggcactattgctgatgttaaggctaatactggtactcgtactagtgctaatgttggcgtta |
| | | gtgatagtgttaaagctaatggtggcgcagatgatgaggagaagaaattaggtctaccttctcatactgataaaccttttgacggtgctttat |
| | | caatatga |
| Non- | Seq | Atgggtgcagacactcctcaacttccagtcatctatctctcggaccaaaccctaaaaccaggaagtgagaagtgggttgaagtgagga |
| func- | ID | gtgatgtccgtaaagctcttgaagactacggcgcttttgaggtgtcatatgatagagtgtcagaagagcttaagaagtcagttttggatgc |
| tional | No | catgatagagcttttcgagttaccagttgaggctaaacagagaaacgtgtctccgaaaccgtacactggatattcaactcataatggtctct |
| 2 | 10 | ctgagagtctagggatccaggattccaatgttttggagaaagttaacgaatttactcaactgctacgccctgattgtgagggtaacaagac |
| | | tatgagcgaaacgatccagaagttttcagagaagttagcagaattggatgtaatggtgagaagaatggtcatagagagctttgggatag |
| | | aaaactactttgatgaacacctcaagtcaacgaattaccgtttgcgactgatgaagtatgtagcaccacctgatgttgatgctaatgttgcg |
| | | gtcagtactaaagattctgttgatggtgctaatacgaatacaactgctaacgctgatgctggtgatattgctaatggtattgctaaagttcata |
| | | ttgatgacgatgctaatgccaagggtgacgtttgcactggtgttggtaccaatcataatggtgatgacgttaatactggtgattgtgctaatg |
| | | ttaagtctaatgtcgacgttggtactaatgttagtgctaaatctagtgttggtgctgatgttaacattggcactattgctgatgttaaggctaat |
| | | actggtactcgtactagtgctaatgttggcgttagtgatagtgttaaagctaatggtggcgcagatgatgaggagaagaaattaggtctac |
| | | cttctcatactgataaaccttttgacggtgctttatcaatatga |

TABLE 3
DNA sequences of Alk genes.

| | | |
|---|---|---|
| Sprout | Seq | ATGGGTGCAGACACTCCTCAACTTCCAGTCATCTATCTCTCGGACCAAACCCTAAAAC |
| | ID | CAGGAGGTGAGAAGTGGGTTGAAGTGAGGAGTGATGTCCGTAAAGCTCTTGAAGACT |
| | No | ACGGCGCTTTTGAGGTGTCATATGATAGAGTGTCAGAAGAGCTTAAGAAGTCAGTTTT |
| | 11 | GGATGCCATGATAGAGCTTTTCGAGTTACCAGTTGAGGCTAAACAGAGAAACGTGTC |
| | | TCCGAAACCTTACACTGGATATTCAACTCATAATGGTCTCTCTGAGAGTCTAGGGATC |
| | | CAGGATTCCAATGTTTTGGAGAAAGTTAACGAATTTACTCAACTGCTACGCCCTGATT |
| | | GTGAGGGTAACAAGACTATGAGGTAACGTCATGTCAAAAAAAAAAAAAAACTCAGT |
| | | AATGCTTGATAGAGTATATTGTTCGTAAATTCCTGTTATTTTTTCATCAAAAATATTTT |
| | | TGTAATTTTTTGTTAGTTGCTTTTATAAGCTATATATATATTGTGACAATTTTCTATATA |
| | | AAACCTTGTTTTTATTTTTATTTTCTTGGAAAAATGTCACTCATATAGTAATGAACTGA |
| | | AATTTTGTTTTCAATCACACGTTTGTCACATTTTATTTTCTTGTTTCTATTAATATACCT |
| | | ATATAAATTTTCTTCGTGTTGAAAACAGCGAAACGATCCAGAAGTTTTCAGAGAAGTT |
| | | AGCAGAATTGGATGTAATGGTGAGAAGAATGGTCATAGAGAGCTTTGGGATAGAAAA |
| | | CTACTTTGATGAACACCTCAAGTCAACGAATTACCGTTTGCGACTGATGAAGTATGTA |
| | | GCACCACCTGATGTTGATGCTAATGTTGCGGTCAGTACTAAAGATTCTGTTGATGGTG |
| | | CTAATACGAATACAACTGCTAACGCTGATGCTGGTGATATTGCTAATGGTATTGCTAA |
| | | AGTTCATATTGATGACGATGCTAATGCCAAGGGTGACGTTTGCACTGGTGTTGGTACC |
| | | AATCATAATGGTGATGACGTTAATACTGGTGATTGTGCTAATGTTAAGTCTAATGTCG |
| | | ACGTTGGTACTAATGTTAGTGCTAAATCTAGTGTTGGTGCTGATGTTAACATTGGCAC |
| | | TATTGCTGATGTTAAGGCTAATACTGGTACTCGTACTAGTGCTACTGTTGGCGTTAGT |
| | | GATAGTGTTAAAGCTAATGGTGGCGCAGATGATGAGGAGAAGAAATTAGGTCTACCT |
| | | TCTCATACTGATAAAAACCTTTTGACGGTGCTTTATCAATATGAGATTGAAGGTTTGG |
| | | AGGTGTTAACCAAAGATGAAAAGTGGATCAGGTTAAAACCATCTCATAACTCTTTCGT |
| | | TGTTATGGCTGGAGATTCTCTATATGTAAGTTCTAACTTATTTTATTTTTAAATGGGAT |
| | | GCACCATAATATATTAATTGCGATCCTCATAATAATCATAACTTTTTTATGATATATAT |
| | | ATTATAACTTTATGATATTCATATTCATAACTTTATGATATAAATATTATATAATTCAC |
| | | TTTTTTATTTTTTCTTATCACTTTCACAAATGCATGTCGACGTTACTAAAGTGTTTATAC |
| | | ATTAACATTATGTTTTCTTCTCATCATGAATCTTGGTGTTGGATTTTTTTTTATTTTTTTT |
| | | TGCAGGCACTTATGAATGGTAGACTATCTCGTCCGTTTCATAGAGTAAGAGTAACGGA |
| | | AAGAAAGAAGACAAGGTATTCAATAGCATTGTTCTCGACTCCAAACGGAGATTACAT |
| | | CATCGAACCACCAAAAGAGCTTGTGGACGAGAAGCACCCACGTCTCTTCAAACCATT |
| | | CACTTTCGTTGACTTGATGAGTTTCTATCACACTGAGGCTGGTCGTAGACCTCGTTCTA |
| | | CTCTTCACGCTTATTGTGCCGTCTCTGGAGCATAA |
| Kale | Seq | ATGGGTGCAGACACTCCTCAACTTCCAGTCATCTATCTCTCGGACCAAACCCTAA |
| Cauli- | ID | AACCAGGAGGTGAGAAGTGGGTTGAAGTGAGGAGTGATGTCCGTAAAGCTCTTG |
| flower | No | AAGACTACGGCGCTTTTGAGGTGTCATATGATAGAGTGTCAGAAGAGCTTAAGAA |
| | 12 | GTCAGTTTTGGATGCCATGATAGAGCTTTTCGAGTTACCAGTTGAGGCTAAACAG |
| | | AGAAACGTGTCTCCGAAACCTTACACTGGATATTCAACTCATAATGGTCTCTCTG |
| | | AGAGTCTAGGGATCCAGGATTCCAATGTTTTGGAGAAAGTTAACGAATTTACTCA |
| | | ACTGCTACGCCCTGATTGTGAGGGTAACAAGACTATGAGGTAACGTCATGTCAAA |
| | | AAAAAAAAAAAACTCAGTAATGCTTGATAGAGTATATTGTTCGTAAATTCCTGTT |
| | | ATTTTTTCATCAAAAATATTTTTGTAATTTTTTGTTAGTTGCTTTTATAAGCTATAT |
| | | ATATATTGTGACAATTTTCTATATAAAACCTTGTTTTTATTTTTATTTTCTTGGAAA |
| | | AATGTCACTCATATAGTAATGAACTGAAATTTTGTTTTCAATCACACGTTTGTCAC |
| | | ATTTTATTTTCTTGTTTCTATTAATATACCTATATAAATTTTCTTCGTGTTGAAAAC |
| | | AGCGAAACGATCCAGAAGTTTTCAGAGAAGTTAGCAGAATTGGATGTAATGGTG |
| | | AGAAGAATGGTCATAGAGAGCTTTGGGATAGAAAACTACTTTGATGAACACCTCA |
| | | AGTCAACGAATTACCGTTTGCGACTGATGAAGTATGTAGCACCACCTGATGTTGA |
| | | TGCTAATGTTGCGGTCAGTACTAAAGATTCTGTTGATGGTGCTAATACGAATACA |
| | | ACTGCTAACGCTGATGCTGGTGATATTGCTAATGGTATTGCTAAAGTTCATATTGA |
| | | TGACGATGCTAATGCCAAGGGTGACGTTTGCACTGGTGTTGGTACCAATCATAAT |
| | | GGTGATGACGTTAATACTGGTGATTGTGCTAATGTTAAGTCTAATGTCGACGTTG |
| | | GTACTAATGTTAGTGCTAAATCTAGTGTTGGTGCTGATGTTAACATTGGCACTATT |
| | | GCTGATGTTAAGGCTAATACTGGTACTCGTACTAGTGCTACTGTTGGCGTTAGTGA |
| | | TAGTGTTAAAGCTAATGGTGGCGCAGATGATGAGGAGAAGAAATTAGGTCTACCT |
| | | TCTCATACTGATAAAAACCTTTTGACGGTACTTTATCAATATGAGATTGAAGGTTT |
| | | GGAGGTGTTAACCAAAGATGAAAAGTGGATCAGGTTAAAACCATCTCATAACTCT |
| | | TTCGTTGTTATGGCTGGAGATTCTCTATATGTAAGTTCTAACTTATTTTATTTTTAA |
| | | ATGGGATGCACCATAATATATTAATTGCGATCCTCATAATAATCATAACTTTTTTA |
| | | TGATATATATATTATAACTTTATGATATACATATTCATAACTTTATGATATAAATA |
| | | TTATATAATTCACCTTTTTATTTTTTCTTATCACTTTCACAAATGCATGTCGACGTT |
| | | ACTAAAGTGTTTATACATTAACATTATGTTTTCTTCTCATCATGAATCTTGGTGTTG |
| | | GATTTTTTTTTATTTTTTTTTGCAGGCACTTATGAATGGTAGACTATCTCGTCCGTT |
| | | TCATAGAGTAAGAGTAACGGAAAGAAAGAAGACAAGGTATTCAATAGCATTGTT |
| | | CTCGACTCCAAACGGAGATTACATCATCGAACCACCAAAAGAGCTTGTGGACGA |
| | | GAAGCACCCACGTCTCTTCAAACCATTCACTTACGTTGACTTGATGAGTTTCTATC |
| | | ACACTGAGGCTGGTCGTAGACCTCGTTCTACTCTTCACGCTTATTGTACCGTCTCT |
| | | GGAGCATAA |
| Cabbage | Seq | ATGGGTGCAGACACTCCTCAACTTCCAGTCATCTATCTCTCGGACCAAACCCTAA |
| | ID | AACCAGGAAGTGAGAAGTGGGTTGAAGTGAGGAGTGATGTCCGTAAAGCTCTTG |
| | No | AAGACTACGGCGGTTTTGAGGTGTCATATGATAGAGTGTCAGAGGAGCTTAAGAA |
| | 13 | GTCAGTTTTGGATGCCATGATAGAGCTTTTCGAGTTACCAGTTGAGGCTAAACAG |
| | | AGAAACGTGTCTCCGAAACCGTACACTGGATATTCAACTCATAATGGTCTCTCTG |
| | | AGAGTCTAGGGATCCAGGATTCCAATGTTTTGGAGAAAGTTAACGAATTTACTCA |
| | | ACTGCTACGCCCTGATTGTGAGGGTAACAAGACTATGAGGTAACGTCATGTCAAA |
| | | AAAAAAAAAAACTCAGTAATGCTTCATAGAGTAAATTAATTGTTCGTAAATTCCT |
| | | GTTAATCTTTCATCAAAAATATTTTCGTAACTTTTTGCTAGTTGTTTTTCATAAGTT |
| | | ATGTGACAAATTTCTATAAAAAAACCTTGTTTTTATCTTTATTTTTTTGGAAAAAT |
| | | GTCAGTTCACTCATATAGTAATGAACTGAAATTTTGTTTTCAATCTCACGTTTCAC |
| | | GCATTTTTTTTTGTTTCTATTAAAATAGCTATATAAATTTTCTTTGTGTTGAAAACA |
| | | GCGAAACGATCCAGAAGTTTTCAGAGAAGTTAGCAGAATTGGATGTAATGGTGA |
| | | GAAGAATGGTCATAGAGAGCTTTGGGATAGAAAACTACTTTGATGAACACCTCAA |
| | | GTCAACGAATTACCGTTTGCGACTGATGAAGTATGTAGCACCACCTGATGTTGAT |
| | | GCTAATGTTGCGGTCAGTACTAAAGATTCTGTTGATGGTGCTAATACGAATACAA |
| | | CTGCTAACGCTGATGCTGGTGATATTGCTAATGGTATTGCTAAAGTTCATATTGAT |
| | | GACGATGCTAATGCCAAGGGTGACGTTTGCACTGGTGTTGGTACCAATCATAATG |
| | | GTGATGACGTTAATACTGGTGATTGTGCTAATGTTAAGTCTAATGTCGACGTTGGT |
| | | ACTAATGTTAGTGCTAAATCTAGTGTTGGTGCTGATGTTAACATTGGCACTATTGC |
| | | TGATGTTAAGGCTAATACTGGTACTCGTACTAGTGCTAATGTTGGCGTTAGTGATA |
| | | GTGTTAAAGCTAATGGTGGCGCAGATGATGAGGAGAAGAAATTAGGTCTACCTTC |
| | | TCATACTGATAAAAACCTTTTGACGGTGCTTTATCAATATGAGATTGAAGGTTTGG |
| | | AGGTGTTAACCAAAGATGAAAAGTGGATCAGGTTAAAACCATCTCATAACTCTTT |
| | | CGTTGTTATGGCTGGAGATTCTCTATATGTAAGTTCTAACTTATTTTATTTTTAAAT |
| | | GGGATGCACCATAATATATTAATTGCGATCCTCATAATAATCATAACTTTTTTATG |
| | | ATATATATATTATAACTTTATGATATTCATATTCATAACTTTATGATATAAATATT |
| | | ATATAATTCACTTTTTTATTTTTTCTTATCACTTTCACAAATGCATGTCGACGTTAC |
| | | TAAAGTGTTTATACATTAACATTATGTTTTCTTCTCATCATGAATCTTGGTGTTGG |
| | | ATTTTTTTTTATTTTTTTTTGCAGGCACTTATGAATGGTAGACTATCTCGTCCGTTT |
| | | CATAGAGTAAGAGTAACGGAAAGAAAGAAGACAAGGTATTCAATAGCATTGTTC |
| | | TCGACTCCAAACGGAGATTACATCATCGAACCACCAAAAGAGCTTGTGGACGAG |
| | | AAGCACCCACGTCTCTTCAAACCATTCACTTTCGTTGACTTGATGAGTTTCTATCA |
| | | CACTGAGGCTGGTCGTAGACCTCGTTCTACTCTTCACGCTTATTGTGCCGTCTCTG |
| | | GAGCATAA |
| Non- | | ATGGGTGCAGACACTCCTCAACTTCCAGTCATCTATCTCTCGGACCAAACCCTA |
| func- | | AAACCAGGAGGTGAGAAGTGGGTTGAAGTGAGGAGTGATGTCCGTAAAGCTCT |
| tional | | TGAAGACTACGGCGCTTTTGAGGTGTCATATGATAGAGTGTCAGAAGAGCTTA |
| 1 | | AGAAGTCAGTTTTGGATGCCATGATAGAGCTTTTCGAGTTACCAGTTGAGGCTA |
| | | AACAGAGAAACGTGTCTCCGAAACCTTACACTGGATATTCAACTCATAATGGTC |
| | | TCTCTGAGAGTCTAGGGATCCAGGATTCCAATGTTTTGGAGAAAGTTAACGAAT |
| | | TTACTCAACTGCTACGCCCTGATTGTGAGGGTAACAAGACTATGAGGTAACGTC |
| | | ATGTCAAAAAAAAAAAAACTCAGTAATGCTTCATAGAGTAAATTAATTGTTCG |
| | | TAAATTCCTGTTAATCTTTCATCAAAAATATTTTCGTAACTTTTTGCTAGTTGTT |
| | | TTTCATAAGTTATGTGACAAATTTCTATAAAAAACCTTGTTTTTATCTTTATTTT |
| | | TTTGGAAAAATGTTAGTTCACTCATATAGTAATGAACTGAAATTTTGTTTTCAA |
| | | TCTCACGTTTCACGCATTTTTTTTTGTTTCTATTAAAATAGCTATATAAATTTTCT |
| | | TTGTGTTGAAAACAGCGAAACGATCCAGAAGTTTTCAGAGAAGTTAGCAGAAT |
| | | TGGATGTAATGGTGAGAAGAATGGTCATAGAGAGCTTTGGGATAGAAAACTAC |
| | | TTTGATGAACACCTCAAGTCAACGAATTACCGTTTGCGACTGATGAAGTATGTA |
| | | GCACCACCTGATGTTGATGCTAATGTTGCGGTCAGTACTAAAGATTCTGTTGAT |
| | | GGTGCTAATACGAATACAACTGCTAACGCTGATGCTGGTGATATTGCTAATGGT |
| | | ATTGCTAAAGTTCATATTGATGACGATGCTAATGCCAAGGGTGACGTTTGCACT |
| | | GGTGTTGGTACCAATCATAATGGTGATGACGTTAATACTGGTGATTGTGCTAAT |
| | | GTTAAGTCTAATGTCGACGTTGGTACTAATGTTAGTGCTAAATCTAGTGTTGGT |
| | | GCTGATGTTAACATTGGCACTATTGCTGATGTTAAGGCTAATACTGGTACTCGT |
| | | ACTAGTGCTAATGTTGGCGTTAGTGATAGTGTTAAAGCTAATGGTGGCGCAGAT |
| | | GATGAGGAGAAGAAATTAGGTCTACCTTCTCATACTGATAAACCTTTTGACGGT |
| | | GCTTTATCAATATGAGATTGAAGGTTTGGAGGTGTTAACCAAAGATGAAAAGT |
| | | GGATCAGGTTAAAACCATCTCATAACTCTTTCGTTGTTATGGCTGGAGATTCTC |
| | | TATATGTAAGTTCTAACTTATTTTATTTTTAAATGGGATGCACCATAATATCTTA |
| | | ATTGCGATCATCATAATAATCATAACTTTTTTATGATATATATTATAACTTTATG |
| | | ATATACATATTCATAACTTTATGATATAAATATTACATAATTCACCTTTTTATTA |
| | | TTTCTTATCACTTTCACAAATGCATGTCGACGTTACTAAAGTGTTTATACATTAA |
| | | CATTATGTTTTCTTCTCATCATGAATCTTGTGTTGGTTTTTTTATTATTATTTTTTT |
| | | GCAGGCACTTATGAATGGTAGACTATCTCGTCCGTTTCATAGAGTAAGAGTAAC |
| | | GGAAAGAAAGAAGACAAGGTATTCAATAGCATTGTTCTCGACTCCAAACGGAG |
| | | ATTACATCATCGAACCACCAAAAGAGCTTGTGGACGAGAAGCACCCACGTCTC |
| | | TTCAAATCATTCACTTACGTTGACTTGATGAGTTTCTATCACACTGAGGCTGGTC |
| | | GTAGACCTCGTTCTACTCTTCACGCTTATTGTGCCGTCTCTGGAGCATAA |
| Non- | Seq | ATGGGTGCAGACACTCCTCAACTTCCAGTCATCTATCTCTCGGACCAAACCCTA |
| func- | ID | AAACCAGGAAGTGAGAAGTGGGTTGAAGTGAGGAGTGATGTCCGTAAAGCTCT |
| tional | No | TGAAGACTACGGCGCTTTTGAGGTGTCATATGATAGAGTGTCAGAAGAGCTTA |
| 2 | 15 | AGAAGTCAGTTTTGGATGCCATGATAGAGCTTTTCGAGTTACCAGTTGAGGCTA |
| | | AACAGAGAAACGTGTCTCCGAAACCGTACACTGGATATTCAACTCATAATGGT |
| | | CTCTCTGAGAGTCTAGGGATCCAGGATTCCAATGTTTTGGAGAAAGTTAACGAA |
| | | TTTACTCAACTGCTACGCCCTGATTGTGAGGGTAACAAGACTATGAGGTAACGT |
| | | CATGTCAAAAAAAAAAAAACTCAGTAATGCTTCATAGAGTAAATTAATTGTTC |
| | | GTAAATTCCTGTTAATCTTTCATCAAAAATATTTTCGTAACTTTTTGCTAGTTGT |
| | | TTTTCATAAGTTATGTGACAAATTTCTATAAAAAACCTTGTTTTTATCTTTATTT |
| | | TTTTGGAAAAATGTTAGTTCACTCATATAGTAATGAACTGAAATTTTGTTTTCA |
| | | ATCTCACGTTTCACGCATTTTTTTTTGTTTCTATTAAAATAGCTATATAAATTTTC |
| | | TTTGTGTTGAAAACAGCGAAACGATCCAGAAGTTTTCAGAGAAGTTAGCAGAA |
| | | TTGGATGTAATGGTGAGAAGAATGGTCATAGAGAGCTTTGGGATAGAAAACTA |
| | | CTTTGATGAACACCTCAAGTCAACGAATTACCGTTTGCGACTGATGAAGTATGT |
| | | AGCACCACCTGATGTTGATGCTAATGTTGCGGTCAGTACTAAAGATTCTGTTGA |
| | | TGGTGCTAATACGAATACAACTGCTAACGCTGATGCTGGTGATATTGCTAATGG |
| | | TATTGCTAAAGTTCATATTGATGACGATGCTAATGCCAAGGGTGACGTTTGCAC |
| | | TGGTGTTGGTACCAATCATAATGGTGATGACGTTAATACTGGTGATTGTGCTAA |
| | | TGTTAAGTCTAATGTCGACGTTGGTACTAATGTTAGTGCTAAATCTAGTGTTGG |
| | | TGCTGATGTTAACATTGGCACTATTGCTGATGTTAAGGCTAATACTGGTACTCG |
| | | TACTAGTGCTAATGTTGGCGTTAGTGATAGTGTTAAAGCTAATGGTGGCGCAGA |
| | | TGATGAGGAGAAGAAATTAGGTCTACCTTCTCATACTGATAAACCTTTTGACGG |
| | | TGCTTTATCAATATGAGATTGAAGGTTTGGAGGTGTTAACCAAAGATGAAAAG |
| | | TGGATCAGGTTAAAACCATCTCATAACTCTTTCGTTGTTATGGCTGGAGATTCT |
| | | CTATATGTAAGTTCTAACTTATTTTATTTTTAAATGGGATGCACCATAATATCTT |
| | | AATTGCGATCATCATAATAATCATAACTTTTTATGATATATATTATAACTTTATG |
| | | ATATACATATTCATAACTTTATGATATAAATATTACATAATTCACCTTTTTATTA |
| | | TTTCTTATCACTTTCACAAATGCATGTCGACGTTACTAAAGTGTTTATACATTAA |
| | | CATTATGTTTTCTTCTCATCATGAATCTTGTGTTGGTTTTTTTATTATTATTTTTTT |
| | | GCAGGCACTTATGAATGGTAGACTATCTCGTCCGTTTCATAGAGTAAGAGTAAC |
| | | GGAAAGAAAGAAGACAAGGTATTCAATAGCATTGTTCTCGACTCCAAACGGAG |
| | | ATTACATCATCGAACCACCAAAAGAGCTTGTGGACGAGAAGCACCCACGTCTC |
| | | TTCAAATCATTCACTTACGTTGACTTGATGAGTTTCTATCACACTGAGGCTGGTC |
| | | GTAGACCTCGTTCTACTCTTCACGCTTATTGTGCCGTCTCTGGAGCATAA |

TABLE 4
Sequences of mutated Alk Proteins.

| | | |
|---|---|---|
| Sprout | Seq | MGADTPQLPVIYLSDQTLKPGGEKWVEVRSDVRKALEDYGAFEVSYDRVSEE |
| | ID | LKKSVLDAMIELFELPVEAKQRNVSPKPYTGYSTHNGLSESLGIQDSNVLEKV |
| | No | NEFTQLLRPDCEGNKTMSETIQKFSEKLAELDVMVRRMVIESFGIENYFDEHLK |
| | 16 | STNYRLRLMKYVAPPDVDANVAVSTKDSVDGANTNTTANADAGDIANGIAK |
| | | VHIDDDANAKGDVCTGVGTNHNGDDVNTGDCANVKSNVDVGTNVSAKSSV |
| | | GADVNIGTIADVKANTGTRTSATVGVSDSVKANGGADDE |
| Kale | Seq | MGADTPQLPVIYLSDQTLKPGGEKWVEVRSDVRKALEDYGAFEVSYDRVSEE |
| or | ID | LKKSVLDAMIELFELPVEAKQRNVSPKPYTGYSTHNGLSESLGIQDSNVLEKV |
| Cauli- | No | NEFTQLLRPDCEGNKTMSETIQKFSEKLAELDVMVRRMVIESFGIENYFDEHLK |
| flower | 17 | STNYRLRLMKYVAPPDVDANVAVSTKDSVDGANTNTTANADAGDIANGIAK |
| | | VHIDDDANAKGDVCTGVGTNHNGDDVNTGDCANVKSNVDVGTNVSAKSSV |
| | | GADVNIGTIADVKANTGTRTSATVGVSDS |
| Cabbage | Seq | MGADTPQLPVIYLSDQTLKPGSEKWVEVRSDVRKALEDYGGFEVSYDRVSEE |
| | ID | LKKSVLDAMIELFELPVEAKQRNVSPKPYTGYSTHNGLSESLGIQDSNVLEKV |
| | No | NEFTQLLRPDCEGNKTMSETIQKFSEKLAELDVMVRRMVIESFGIENYFDEHLK |
| | 18 | STNYRLRLMKYVAPPDVDANVAVSTKDSVDGANTNTTANADAGDIANGIAK |
| | | VHIDDDANAKGDVCTGVGTNHNGDDVNTGDCANVKSNVDVGTNVSAKSSV |
| | | GADVNIGTIADVKANTGTRTSANVGVSDS |

### Example 2. Sinigrin and Progoitrin Measurements

### 1. Sampling.

Brussels sprouts were sampled by taking 5 sprouts picked at a random fashion from the stem. Plants that grew on the borders were not sampled as they had more space and nutrition, which could influence the glucosinolate content in the sprouts due to different availability of nutrients such as nitrate. To help the subsequent freeze drying step, the picked sprouts were cut into smaller pieces. 50 gram of fresh material was taken to obtain 5 grams of freeze dried material.

### 2. Freeze Drying.

Fresh material was frozen in freezer at −20° C. and subsequently freeze dried under vacuum in an Edwards freeze dryer. To avoid enzymatic break down of glucosinolates, care was taken not to defrost the samples in the process.

### 3. Measurement.

LC-MS was used to determine the glucosinolate levels in the samples. The detection limit was 3 micromole per 100 gram fresh material. The measurement error is according to the norm SANCO/12571/2013 E13 https://www.eurl-pesticides.eu/library/docs/allcrl/AqcGuidance_Sanco_2013_12571.pdf.

Standards of sinigrin and progoitrin were used for identification and quantification of LC-MS peaks.

TABLE 5
Measurement of sinigrin and progoitrin levels -
plants according to the invention.

| | | | | Sum |
|---|---|---|---|---|
| | | | | progoitrin + |
| | | Progoitrin | Sinigrin | sinigrin |
| | | (micromole/ | (micromole/ | (micromole/ |
| | | 100 gr fresh | 100 gr fresh | 100 gr fresh |
| | | weight) | weight) | weight) |
| deposit | plant 1 | 0 | 0 | 0 |
| NCIMB | plant 2 | 0 | 0 | 0 |
| 44053* | plant 3 | 0 | 0 | 0 |
| | plant 4 | 0 | 0 | 0 |
| | plant 5 | 0 | 0 | 0 |
| | plant 6 | 0 | 0 | 0 |
| | plant 7 | 0 | 0 | 0 |
| | plant 8 | 0 | 0 | 0 |
| Control** | Control plant 1 | 84 | 131 | 215 |
| | Control plant 2 | 81 | 133 | 214 |
| | Control plant 3 | 77 | 126 | 203 |
| | Control plant 4 | 64 | 121 | 185 |

A value of zero means the measurement is under the detection limit (3 micromole/100 gram fresh weight or less)
*Deposit NCIMB 44053 - for this measurement plants were selected where the non-functional ALK gene was present homozygously.
**Control plants: Plants with the same genetic background as the deposit but with a functional ALK gene.

### Comparator Varieties

Measurements with selected known varieties were per performed for comparison. In this measurement 5 plans of each variety were measured.

TABLE 6
Sum of sinigrin and progoitrin levels
in known varieties of Brussels sprouts.

| | Sum |
|---|---|
| | sinigrin + progoitrin |
| | (micromole/100 gram fresh weight) |
| Deposit NCIMB 44053 | 0 |
| (for comparison) | |
| Davlin F1 | 177 |
| Hestia F1 | 143 |
| Franklin F1 | 169 |
| Dagan F1 | 116 |
| Marte F1 | 221 |
| Irene F1 | 163 |
| Divino F1 | 97 |
| Attis F1 | 61 |
| Dyonisus F1 | 152 |
| Octia F1 | 86 |
| Dimitri F1 | 125 |
| Nautic F1 | 158 |
| Diablo F1 | 244 |
| Doric F1 | 397 |
| Helios F1 | 331 |
| Sofia F1 | 80 |
| Steadia F1 | 91 |
| Neptuno F1 | 143 |
| Faunus F1 | 280 |
| Pontus F1 | 252 |
| Thor F1 | 270 |
| Hemera F1 | 341 |
| Hector F1 | 217 |

### Example 3. CRISPR/Cas9 Knockout of ALK

To assess whether the ALK gene is indeed a functional gene for glucosinolate synthesis in Brussels sprouts, a knockout of ALK was made using CRISPR/Cas9 in a Brussels sprout plant comprising an active ALK gene encoding for a protein having the sequence Seq ID 1. Four target sites were identified (SEQ ID Nos. 19 to 23) and used to generate guide RNAs (gRNAs). The aim of this modification was to introduce a mutation that will result in a truncated protein. The mutation(s) can be either insertion, deletion or another modification. The CRISPR machinery, gRNA and Cas9 proteins, were delivered in Brussels sprouts plant cells, via either PEG mediated transfection of ribonucleoproteins (RNP), or Agrobacterium-mediated gene transfer. Edited cells were regenerated to obtain viable plants. Plants that contained a mutation in the Alk gene were selfed to obtain Brussels sprout plants where the defective gene is present homozygously. Brussels sprout plants without an active Alk gene showed the expected phenotype.

TABLE 7
Identified target sites for generating CRISPR/Cas9
knockouts of ALK, excluding PAM sites.

| Seq ID No. | Sequence |
|---|---|
| Seq ID No 19 | AGAGCTTAAGAAGTCAGTTT |
| Seq ID No 20 | CTCTGAGAGTCTAGGGATCC |
| Seq ID No 21 | AGGTTTTTATCAGTATGAGA |
| Seq ID No 22 | GTTTCATAGAGTAAGAGTAA |
| Seq ID No 23 | ATTGATAAAGTACCGTCAAA |

### Example 4. Taste Trial of the Brussels Sprouts

In total 243 people participated in the taste trial. The test was carried out on the premises of Bejo Zaden in Warmenhuizen, the Netherlands, among the employees of the company. Each of the participants got to taste two sprouts, one labelled ‘x’ the other one labelled ‘y’. Brussels sprouts labelled ‘x’ were plants according to the invention as in the Deposit NCIMB 44053, with a defective ALK gene. Brussels sprouts labelled ‘y’ have the same genetics as ‘x’, except that sprouts “y” have a functional ALK gene and are able to produce sinigrin and progoitrin, similarly to traditional Brussels sprouts. The participants were not informed on the difference between ‘x’ and ‘y’. The participants had to answer the question: ‘Which sprout tastes better?’, the options for answer were: ‘x’, ‘y’ or ‘no difference’. A majority of the participants (145/243, 60%) had a preference for Brussels sprouts labelled ‘x’, see Table 7 below.

Participants often mentioned that ‘x’ had softer/sweeter taste.

TABLE 8
Results of the taste trial.

| Preference | Nr of people |
|---|---|
| ‘x’ | 146 |
| ‘y’ | 85 |
| No difference | 12 |

### Example 5. Identification of an ALK Mutant in a TILLING Population

To assess whether ALK is indeed a functional gene for glucosinolate synthesis in Brussels sprouts, an ALK mutant allele can be found in a Brussels sprouts TILLING population by screening and/or resequencing. The TILLING population was generated by treating seeds of a Brussels sprouts with Ethyl Methyl Sulfate (EMS). Surviving seedlings were grown into plants and subsequently selfed to obtain M2 seeds. The M2 seeds can be sampled to identify ALK mutants. Plants that have a nonsense mutation in ALK can be selected and backcrossed several times, while selecting for the mutation. This will result in Brussels sprouts with a defect in the glucosinolate synthesis pathway, namely in the in the step of production sinigrin due to the absence of active ALK gene.

1. A plant, wherein the plant substantially does not, or does not, comprise sinigrin and progoitrin, wherein the genome of the plant comprises a gene encoding an enzymatically inactive 2-oxa-acid dependent dioxygenase (ALK) protein, said enzymatically inactive ALK protein comprises one or more mutations as compared to
SEQ ID No. 1 wherein the plant is Brussels sprout; or
SEQ ID No. 2 wherein the plant is kale or cauliflower; or
1 SEQ ID No. 3 wherein the plant is cabbage.

2. The plant according to claim 1, wherein the one or more mutations are selected from the group consisting of insertions, deletions, frame shifts, substitutions, and truncations in
SEQ ID No. 1 wherein the plant is Brussels sprout; or
SEQ ID No. 2 wherein the plant is kale or cauliflower; or
SEQ ID No. 3 wherein the plant is cabbage.

3. The plant according to claim 1, wherein the genome of the plant comprises a gene encoding an enzymatically inactive 2-oxa-acid dependent dioxygenase (ALK) protein, said enzymatically inactive ALK protein has the amino acid sequence of
SEQ ID No. 16, or amino acid sequences having at least 90% sequence identity therewith, wherein the plant is Brussels sprout; or
SEQ ID No. 17, or amino acid sequences having at least 90% sequence identity therewith, wherein the plant is kale or cauliflower; or
SEQ ID No. 18, or amino acid sequences having at least 90% sequence identity therewith, wherein the plant is cabbage.

4. The plant according to claim 1, wherein the gene encoding an enzymatically inactive 2-oxa-acid dependent dioxygenase (ALK) protein is homozygously present in the genome of the plant.

5. The plant according to claim 1, wherein the plant does not comprise a gene encoding an enzymatically active 2-oxa-acid dependent dioxygenase (ALK) protein of:
SEQ ID No. 1 wherein the plant is Brussels sprout; or
SEQ ID No. 2 wherein the plant is kale or cauliflower; or
SEQ ID No. 3 wherein the plant is cabbage.

6. The plant according to claim 1, wherein the gene encoding an enzymatically inactive 2-oxa-acid dependent dioxygenase (ALK) protein is obtainable from, obtained from, or is from a plant whereof representative seeds are deposited under deposit number NCIMB 44053.

7. The plant according to claim 1, wherein the plant is a cytoplasmic male sterile (CMS) plant and/or a hybrid plant.

8. Seeds, progeny, edible parts, egg cells, callus, suspension culture, somatic embryos, clones, embryos, or plant parts, or cells of the plant according to claim 1.

9. A method for providing a plant according to claim 1,
wherein the method comprises the step of introducing one or more mutations in a gene encoding an enzymatically active 2-oxa-acid dependent dioxygenase (ALK) protein to provide a plant that substantially does not comprise sinigrin and progoitrin.

10. The method according to claim 9, wherein the one or more mutations in the gene encoding an enzymatically active 2-oxa-acid dependent dioxygenase (ALK) protein are provided by genome editing, CRISPR Cas or mutagenesis.

11. A method for providing a plant according to claim 1, wherein the method comprises the step of:
(i) providing a Brassica plant comprising a gene encoding an enzymatically active 2-oxa-acid dependent dioxygenase (ALK) protein;
(ii) crossing the plant of step (i) with a Brassica plant comprising a gene encoding an enzymatically inactive 2-oxa-acid dependent dioxygenase (ALK) protein;
(iii) optionally, selfing the plant obtained in step ii) at least once; and
(iv) selecting a plant homozygously comprising in its genome a gene encoding an enzymatically inactive 2-oxa-acid dependent dioxygenase (ALK) protein.

12. The method according to claim 11, wherein a Brassica plant comprising a gene encoding an enzymatically inactive 2-oxa-acid dependent dioxygenase (ALK) protein of step (ii) is, or is derived from, a plant whereof representative seeds are deposited under deposit number NCIMB 44053.

13-15. (canceled)
